(19) United States
(12) Patent Application Publication
(10) Pub. No.: US 2006/0051404 A1
(43) Pub. Date: Mar. 9, 2006

(54) Microstructure roller system

(75) Inventors: Yehoshua Yeshurun, HAIFA (IL); Meir Hefetz, Harashim (IL); Gil Fruchtman, Kiryat Tivon (IL); Yoel Sefi, Kibutz Malkia (IL); Yotam Levine, Nes Ziona (IL)

Correspondence Address: Mark Friedman;Bill Polkinhorn, 9003 Florin Way, Upper Marlboro, MD, 20772, US

(73) Assignee: NANO PASS TECHNOLOGIES LTD., HAIFA (IL)

(21) Appl. No.: 10/535,252

(22) PCT Filed: Nov. 18, 2003
(86) PCT No.: PCT/IL03/00974

§ 371 Date: May 18, 2005

(30) Foreign Application Priority Data
Nov. 18, 2002 (IL) 152912

(51) Int. Cl. A61K 9/70

(52) U.S. Cl. 424/449

(57) ABSTRACT

A microstructure for transferring a substance through the surface of the skin, comprising a substrate having a first and a second side and at least one microstructure projecting from the second side of the substrate. The micrstructure has at least one hollow. The hollow is isolated from the fluid connection with the first side of the substrate. The hollow is configured such that, when the microstructure is inserted through the surface of the skin, at least part of the substance is transferred through the surface of the skin in the hollow.

### FIELD AND BACKGROUND OF THE INVENTION

The present invention relates to microstructures and, in particular, it concerns microstructures used to apply cosmetics as well as to deliver drugs and vaccinations.

By way of introduction, the successful application of cosmetics and medical skin treatments, for example cosmetic and medicinal creams, depends on the skin penetration of the cosmetic or medicine. It is known in the art of cosmetic and medical skin treatments to use microstructures to penetrate the surface of the skin to apply substances to the stratum corneum and deeper in the skin. The term “microstructures” is defined herein as, a structure used to penetrate the surface of the skin. Examples of microstructures include, but are not limited to, microneedles and micro-pyramids. Microstructures are typically used to accelerate growth of collagen and thus soften skin lines, improve healing and scarring, restore skin tightening (anti-aging effect) and thicken skin. In addition, microstructures are used to enhance penetration of active compounds applied before, during or after application of the microstructures.

The prior art, in particular PCT publications WO/0,247,555 and WO/0,249,711, teach using a roller apparatus having small steel pins protruding from the roller head. The steel pins are individually set into the roller head. In use, the roller head is rolled over skin in order to create channels in the skin. A shortcoming of the aforementioned system is that the spacing between pins is limited due to the construction method of setting each pin into the roller head and overcrowding within the roller head as the steel pins extend into the roller head limiting the spacing of the pins to 3 mm in a direction of curvature of the head and to 2 mm in a transverse direction on the head surface. This space limitation reduces the effectiveness of the treatment as treatment improves when the holes are closer together. Another shortcoming of the aforementioned system is that pressing very hard on the roller may result in dislocation of the steel pins from the roller head or much deeper penetration than is desired for the application. Therefore, safety is compromised, especially for cosmetic applications where it is essential to ensure that the dermis in not penetrated. A further shortcoming of the aforementioned system is that when used in combination with a medical or cosmetic substance in gel, cream or another form, the needles cannot be used to effectively transfer the substance into the skin. The needles can only be used as a pre treatment or less-efficient post-treatment.

Therefore there is a need for a system to safely and effectively transfer an active medical or cosmetic substance into the skin at the appropriate level, for example, cosmetic compounds into or just under the stratum corneum, vaccines into the epidermis and drugs for systemic and local distribution into the dermis.

### SUMMARY OF THE INVENTION

The present invention is a microstructure roller system construction and method of operation thereof.

According to the teachings of the present invention there is provided, a microstructure structure for transferring a substance through the surface of the skin, comprising: (a) a substrate having a first side and a second side; and (b) a plurality of microstructures projecting from the second side of the substrate, each of the microstructure having at least one hollow, the hollows being isolated from fluid connection with the first side of the substrate, the hollows being configured such that, when the microstructures are inserted through the surface of the skin, at least part of the substance is transferred through the surface of the skin in the hollows.

According to a further feature of the present invention, the hollows is a single-opening hollows.

According to a further feature of the present invention, each of the microstructures has a plurality of hollows isolated from fluid connection with the first side of the substrate.

According to the teachings of the present invention there is also provided a medical substance disposed in the hollows.

According to the teachings of the present invention there is also provided a cosmetic substance disposed in the hollows.

According to the teachings of the present invention there is also provided a rolling arrangement, wherein the rolling arrangement, the substrate and the microstructures together form a microstructure rolling system, the microstructure rolling system being configured such that, at least part of the substance is transferred through the surface of the skin as the microstructure rolling system is rolled over the surface of the skin.

According to a further feature of the present invention, the microstructure rolling system includes a dispensing arrangement configured to store the substance and to dispense the substance into the hollows of the microstructures.

According to a further feature of the present invention, there is also provided a microstructure structure for transferring a substance through the surface of the skin, comprising: (a) a substrate having a first side and a second side; and (b) at least one microstructure projecting from the second side of the substrate, the at least one microstructure having at least one hollow, the at least one hollow being isolated from fluid connection with the first side of the substrate, the at least one hollow being configured such that, when the at least one microstructure is inserted through the surface of the skin, at least part of the substance is transferred through the surface of the skin in the at least one hollow.

According to a further feature of the present invention, the hollow is a single-opening hollow.

According to a further feature of the present invention, the at least one microstructure has a plurality of hollows, the hollows being isolated from fluid connection with the first side of the substrate.

According to a further feature of the present invention, there is also provided a method for transferring a substance through the surface of the skin using a microstructure structure, the microstructure structure including a substrate having a first side and a second side, the microstructure structure including a plurality of microstructures projecting from the second side of the substrate, the method including the steps of: (a) applying the substance to the microstructure structure from the second side; and (b) disposing the microstructure structure onto the surface of the skin, such that the microstructures penetrate the surface of the skin thereby transferring at least part of the substance through the surface of the skin.

According to a further feature of the present invention: (a) the step of applying is performed by disposing at least part of the substance into hollows of the microstructures, the hollows being isolated from fluid connection with the first side of the substrate; and (b) the step of disposing the microstructure structure onto the surface of the skin is performed such that at least part of the substance is transferred through the surface of the skin in the hollows.

According to a further feature of the present invention, the substance is a dry substance.

According to a further feature of the present invention, the substance is a gel.

According to a further feature of the present invention, the substance is a cream.

According to a further feature of the present invention, the substance is a cosmetic substance.

According to a further feature of the present invention, the substance is a medical substance.

According to a further feature of the present invention, there is also provided a microstructure system for penetrating the surface of the skin, comprising: (a) a rolling element; (b) a plurality of microstructures interconnected with the rolling element, the microstructures having an inter-needle spacing of less than 1 mm; and (c) a handle mechanically connected to the rolling element, the rolling element being configured to rotate with respect to the handle, wherein the rolling element, the microstructures and the handle are configured such that, when the handle is manipulated in order to roll the microstructures over the surface of the skin, the microstructures penetrate the surface of the skin.

According to a further feature of the present invention, there is also provided a microstructure system for penetrating the surface of the skin, comprising: (a) a rolling element; (b) an integrally formed microstructure structure having a substrate and a plurality of microstructures projecting from the substrate, the microstructure structure being interconnected with the rolling element; and (c) a handle mechanically connected to the rolling element, the rolling element being configured to rotate with respect to the handle, wherein the rolling element, the microstructure structure and the handle are configured such that, when the handle is manipulated in order to roll the microstructures over the surface of the skin, the microstructures penetrate the surface of the skin.

According to a further feature of the present invention, there is also provided a microstructure system for penetrating the surface of the skin, comprising: (a) a rolling element, the rolling element being substantially spherical; (b) a plurality of microstructures interconnected with the rolling element; (c) and a handle mechanically connected to the rolling element, the rolling element being configured to rotate with respect to the handle, wherein the rolling element, the microstructures and the handle are configured such that, when the handle is manipulated in order to roll the microstructures over the surface of the skin, the microstructures penetrate the surface of the skin.

According to a further feature of the present invention, there is also provided a microstructure system for transferring a substance through the surface of the skin, comprising: (a) a rolling element; (b) a plurality of microstructures interconnected with the rolling element; (c) a handle mechanically connected to the rolling element, the rolling element being configured to rotate with respect to the handle; and (d) a dispensing arrangement mechanically connected to the rolling element, the dispensing arrangement being configured to store the substance and to dispense the substance onto the microstructures, wherein the rolling element, the microstructures, the handle and the dispensing arrangement are configured such that, when the handle is manipulated in order to roll the microstructures over the surface of the skin, at least part of the substance is transferred through the surface of the skin.

According to a further feature of the present invention, there is also provided a microstructure system for transferring a substance through the surface of the skin, comprising: (a) a rolling element; (b) a plurality of microstructures interconnected with the rolling element, each of the microstructures having a channel therethrough; (c) a reservoir configured for storing the substance, the reservoir being interconnected with the channels of the microstructures such that, the reservoir is configured for dispensing the substance via the channels of the microstructures; and (d) a handle mechanically connected to the rolling element, the rolling element being configured to rotate with respect to the handle, wherein the rolling element, the microstructures, the reservoir and the handle are configured such that, when the handle is manipulated in order to roll the microstructures over the surface of the skin thereby causing the microstructures to penetrate the surface of the skin, at least part of the substance is transferred through the surface of the skin via the channels of the microstructures.

According to a further feature of the present invention, there is also provided a microstructure system for penetrating the surface of the skin, comprising: (a) a roller; (b) a handle mechanically connected to the roller, the roller being configured to rotate with respect to the handle; aid (c) a plurality of microstructures interconnected with the roller, the microstructures being configured such that: when the roller is rolled in a first direction the microstructures penetrate through the surface of the skin to a first depth; and when the roller is rolled in a second direction the microstructures penetrate through the surface of the skin to a second depth, wherein the first depth is greater than the second depth.

According to a further feature of the present invention, there is also provided according to a further feature of the present invention, the second depth is zero.

### BRIEF DESCRIPTION OF THE DRAWINGS

The invention is herein described, by way of example only, with reference to the accompanying drawings, wherein:

FIG. 1a is an isometric view of a microstructure roller system that is constructed and operable in accordance with a preferred embodiment of the present invention;

FIG. 1b is a side view of the microstructure roller system of FIG. 1a;

FIG. 1c is a top view of the microstructure roller system of FIG. 1a;

FIG. 1d is an expanded view of the region marked by the letter A in FIG. 1a;

FIG. 1e is a schematic cross-sectional view of the microstructure roller system of FIG. 1a;

FIG. 1f is an expanded view of the region marked by the letter A in FIG. 1e;

FIG. 1g is a side view of a microstructure roller system that is constructed and operable in accordance with an alternate embodiment of the present invention;

FIG. 1h is an expanded cross-sectional view of the region marked by the letter A in FIG. 1g;

FIG. 2a is schematic cross-sectional view showing the penetration of the microstructures of the microstructure roller system of FIG. 1a when the roller is rolled in a first direction;

FIG. 2b is schematic cross-sectional view showing the penetration of the microstructures of the microstructure roller system of FIG. 1a when the roller is rolled in a second direction;

FIG. 3 is a schematic view of a microstructure structure of the microstructure roller system of FIG. 1a;

FIG. 4a is an isometric view of a cupped microstructure for use with the microstructure roller system of FIG. 1a;

FIG. 4b is a cut away view of the cupped microstructure of FIG. 4a;

FIG. 4c is an isometric view of an asymmetric cupped microstructure for use with the microstructure roller system of FIG. 1a;

FIG. 4d is a cut away view of the asymmetric cupped microstructure of FIG. 4c;

FIG. 5 is a cross-sectional view of a cut cupped microstructure for use with the microstructure roller system of FIG. 1a;

FIG. 6a is a top view of a pyramidal microstructure having a hollow with a hexagonal cross-section for use with the microstructure roller system of FIG. 1a;

FIG. 6b is a top view of a pyramidal microstructure having two hollows of oval cross-section for use with the microstructure roller system of FIG. 1a;

FIG. 6c is a top view of a pyramidal microstructure having a hollow with a triangular cross-section for use with the microstructure roller system of FIG. 1a;

FIG. 6d is a top view of a pyramidal microstructure having a wide hollow with an oval cross-section for use with the microstructure roller system of FIG. 1a;

FIG. 6e is a top view of a pyramidal microstructure having a narrow hollow with an oval cross-section for use with the microstructure roller system of FIG. 1a;

FIG. 7a is a top view of a first microstructure having a hollow with a circular cross-section for use with the microstructure roller system of FIG. 1a;

FIG. 7b is a top view of a second microstructure having a hollow with a circular cross-section for use with the microstructure roller system of FIG. 1a;

FIG. 7c is a top view of a third microstructure having a hollow with a circular cross-section for use with the microstructure roller system of FIG. 1a;

FIG. 8a is a top view of a pyramidal microstructure having a hollow with a square cross-section for use with the microstructure roller system of FIG. 1a;

FIG. 8b is a cross-sectional view of the microstructure of FIG. 8a;

FIG. 9a is a cross-sectional view of a microstructure having a plurality of hollows thereon for use with the microstructure roller system of FIG. 1a;

FIG. 9b is a side view of the microstructure of FIG. 9a;

FIG. 10a is an isometric view of a microstructure ball-roller system that is constructed and operable in accordance with a second alternate embodiment of the present invention;

FIG. 10b is an expanded view of the region marked by the letter A in FIG. 10a; and

FIG. 10c is a side view of the microstructure ball-roller system of FIG. 10a.

### DESCRIPTION OF THE PREFERRED EMBODIMENTS

The present invention is a microstructure roller system and method of operation thereof.

The principles and operation of a microstructure roller system according to the present invention may be better understood with reference to the drawings and the accompanying description.

The constructions of the present invention are particularly suited for applying cosmetic or delivering drugs and/or vaccinations or other similar compounds into the skin, for example, for delivering cosmetics at very shallow depths of up to 100 micron, for delivering vaccines up to 200 microns and delivering dermatological and systemic related compounds up to 400 microns.

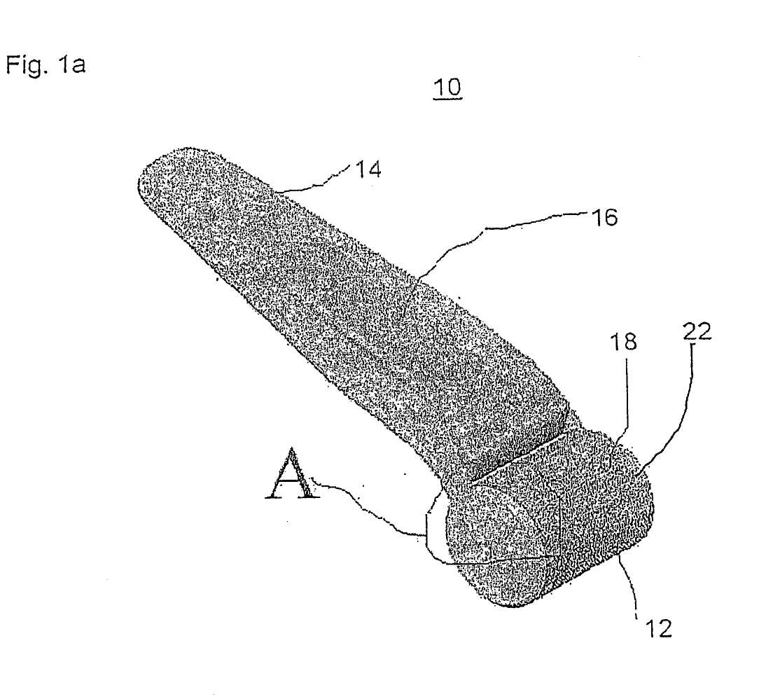

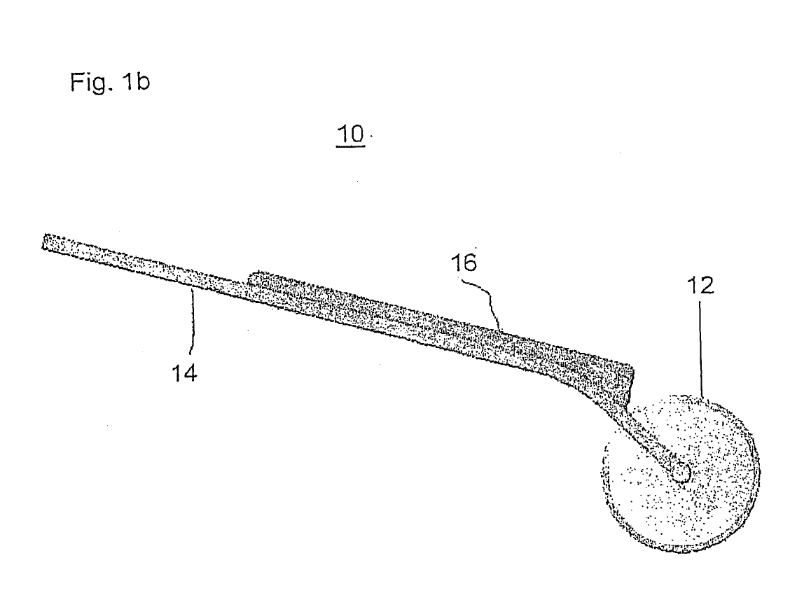

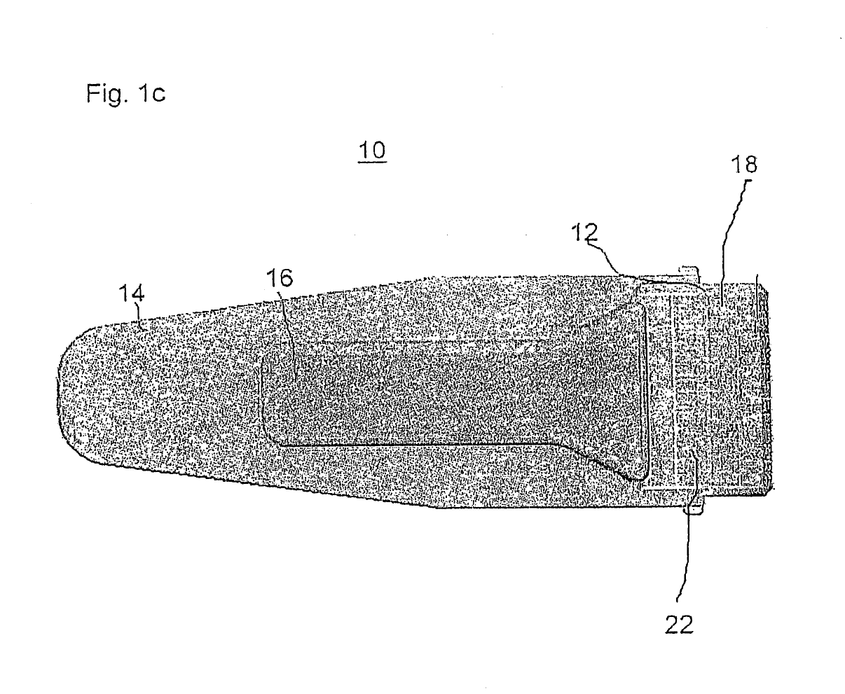

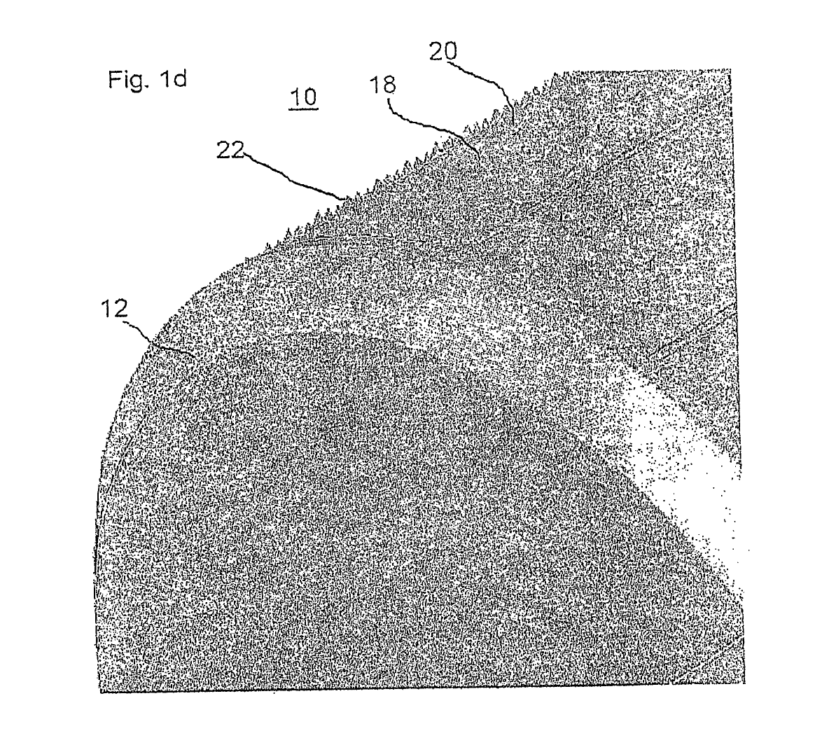

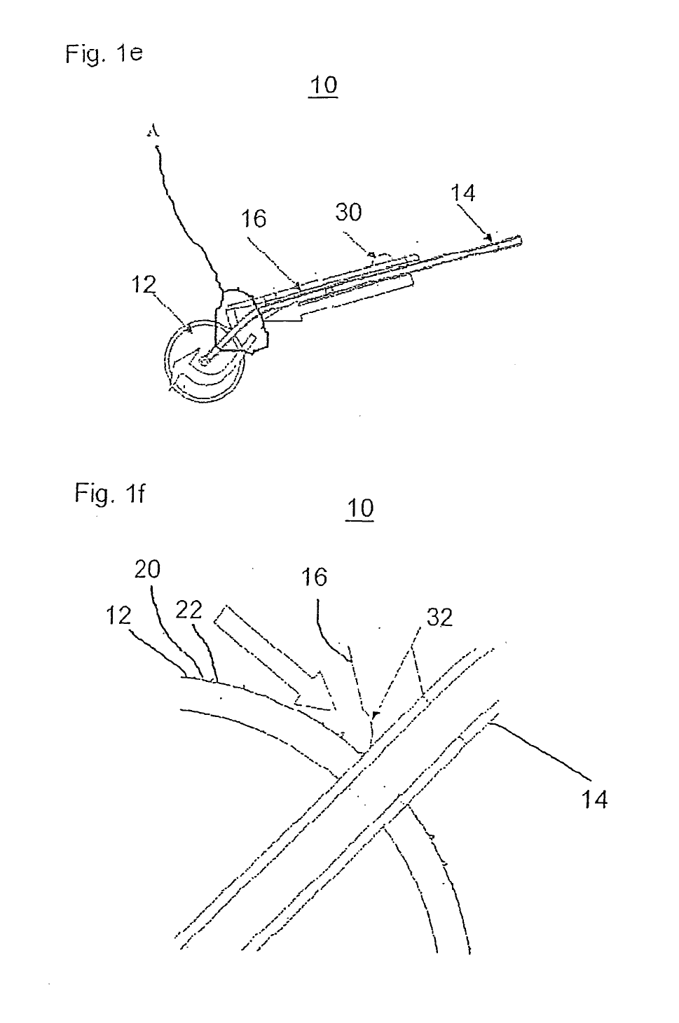

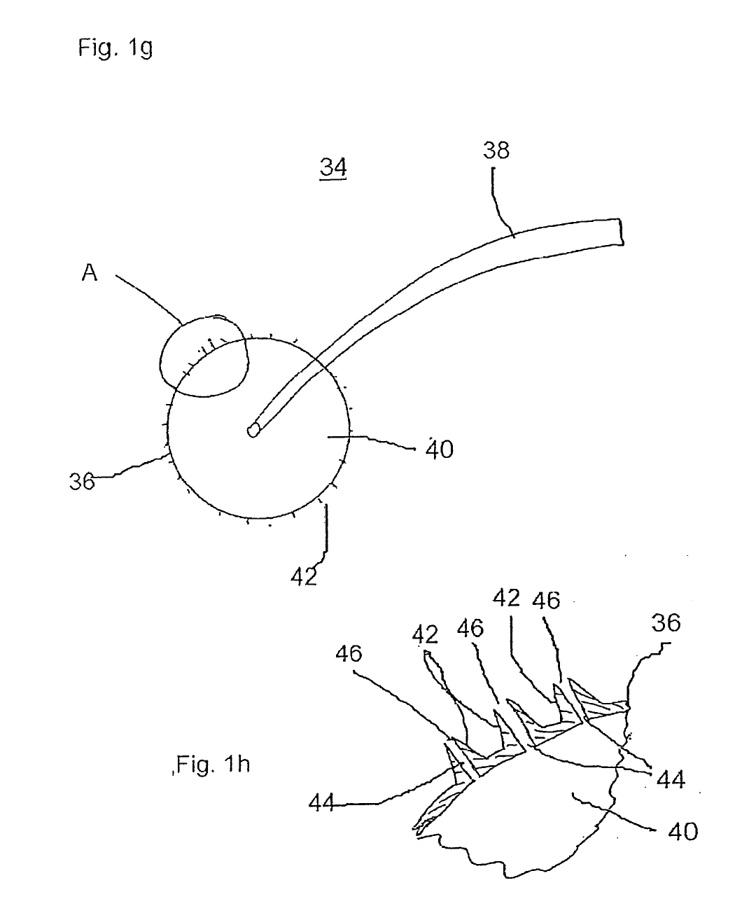

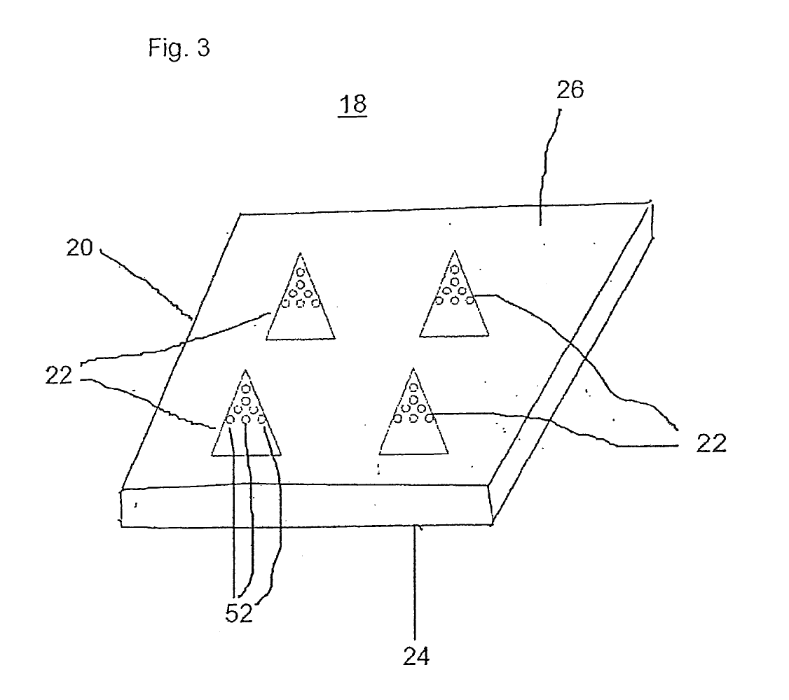

Reference is now made to FIGS. 1a to 1f, which are various views of a microstructure roller system 10 that is constructed and operable in accordance with a preferred embodiment of the present invention. FIG. 1a is an isometric view of microstructure roller system 10. FIG. 1b is a side view of microstructure roller system 10. FIG. 1c is a top view of microstructure roller system 10. FIG. 1d is an expanded view of the region marked by the letter A in FIG. 1a. FIG. 1e is a schematic cross-sectional view of microstructure roller system 10. FIG. 1f is an expanded view of the region marked by the letter A in FIG. 1e. Microstructure roller system 10 includes a rolling element 12, a handle 14, a dispensing arrangement 16 and a plurality of integrally formed microstructure structures 18. Rolling element 12 is typically has a cylindrical shape. Each integrally formed microstructure structure 18 includes a substrate 20 and a plurality of microstructures 22 (FIG. 1d). Substrate 20 has a first major surface 24 and a second major surface 26 (FIG. 3). Microstructures 22 project from second major surface 26 of substrate 20. Integrally formed microstructure structure 18 is described in more detail with respect to FIG. 3. Integrally formed microstructure structure 18 is described as integrally formed in that substrate 20 and microstructures 22 are typically formed as a single unit. Integrally formed microstructure structures 18 are interconnected with rolling element 12, typically by mounting substrate 20 onto the surface of rolling element 12 such that, microstructures 22 project away from rolling element 12. It will be appreciated by those ordinarily skilled in the art that rolling element 12 and integrally formed microstructure structures 18 can be formed as one integrally formed unit. Microstructures 22 are not individually set into rolling element 12. Therefore, microstructures 22 can be spaced closer together than the pins of prior art roller systems. It is known in the art that silicon and polymer microstructures can be manufactured having an inter-needle spacing of less than 100 microns. Therefore, microstructures 22 generally have an inter-needle spacing of less than 1 mm, preferably 450 microns, thereby increasing the effectiveness of the treatment performed by microstructure roller system 10. The term, “inter-needle spacing” is defined herein as, the shortest straight-line distance between the outside edge of a microstructure 22 and the outside edge of the nearest neighboring microstructure 22 measured where microstructures 22 meet substrate 20. Microstructures 22 have a typical length of between 20 and 750 microns depending on the application. Microstructures 22 have a typical base width up to 300 microns, preferably 100 microns. The ratio of the base width to height is typically between 1:1 to 1:5. Rolling element 12 is typically formed from plastic materials (such as polycarbonate or PMMA), Teflon, metal such as stainless steel or ceramics such as glass or alumina. When rolling element 12 is formed from plastic, rolling element 12 is typically formed by injection molding or casting.

Handle 14 is mechanically connected to rolling element 12 such that, rolling element 12 is configured to rotate with respect to handle 14. Rolling element 12, integrally formed microstructure structures 18 and handle 14 are configured such that, when handle 14 is manipulated in order to roll microstructures 22 over the surface of the skin, microstructures 22 penetrate the surface of the skin.

Dispensing arrangement 16 is mechanically connected to rolling element 12. Dispensing arrangement 16 is configured to store a substance (generally medical or cosmetic), typically under pressure, and to dispense the substance onto microstructures 22. The terms “dispense onto microstructures”, “dispose on microstructures” and “apply the substance to the microstructure structure” are defined herein to include disposing the substance into hollows of the microstructures 22. Microstructures 22 with hollows are described in more detail with reference to FIGS. 3 to 9b. However, it will be appreciated by those ordinarily skilled in the art that microstructures without hollows can also be used for microstructures 22. The hollows increase the surface area between the substance and the penetrated skin. Additionally, the hollows increase the amount of substance that each microneedle 22 holds. Therefore, the substance is applied to microstructures 22 from second major surface 26 of substrate 20 as opposed to applying the substance from first major surface 24 through channels in microstructures 22. Dispensing arrangement 16 has an opening 28 (FIG. 1f) close to where dispensing arrangement 16 and rolling element 12 meet. The substance is released from dispensing arrangement 16 by pressing a button 30. The substance is spread onto microstructures 22 by a resilient spreading strip 32. Therefore, microstructure roller system 10 is configured such that, when handle 14 is manipulated in order to roll microstructures 22 over the surface of the skin, the substance is transferred through the surface of the skin by the microstructures 22. When microstructures 22 have hollows, the substance is transferred through the surface of the skin mainly in the hollows of microstructures 22. The term “transferred through the surface of the skin” is defined herein to include transferring the substance from outside the body into any layer of the skin, for example, but not limited to, the stratum corneum, the epidermis and the dermis. Also, the term “penetrate the surface of the skin” is defined herein as penetrating the skin from outside the body by a microstructure which reaches any layer of the skin, for example, but not limited to, the stratum corneum, the epidermis and the dermis. It will be appreciated those ordinarily skilled in the art that dispensing arrangement 16 can be implemented as a standalone reservoir having a flexible tube attached to handle 14 for feeding rolling element 12 with the substance. This arrangement may be suitable for mass treatment in some clinics.

Reference is now made to FIGS. 1g and 1h. FIG. 1g is a side view of a microstructure roller system 34 that is constructed and operable in accordance with an alternate embodiment of the present invention. FIG. 1h is an expanded cross-sectional view of the region marked by the letter A in FIG. 1g. microstructure roller system 34 includes a rolling element 36, a handle 38, a reservoir 40 and a plurality of microstructures 42. Rolling element 36 is typically a cylindrical roller. Handle 38 is mechanically connected to rolling element 36 such that, rolling element 36 is configured to rotate with respect to handle 38. Reservoir 40 is disposed within rolling element 36. Reservoir 40 is configured for storing a substance. Microstructures 42 are interconnected with rolling element 36. Each microstructure 42 has a channel 44 therethrough. Each channel 44 is interconnected with reservoir 40 and an opening 46 on a respective microstructure 42 such that, reservoir 40 is configured for dispensing the substance via channels 44. Therefore, when handle 38 is manipulated in order to roll microstructures 42 over the surface of the skin thereby causing microstructures 42 to penetrate the surface of the skin, at least part of the substance is transferred through the surface of the skin via channels 44 of microstructures 42.

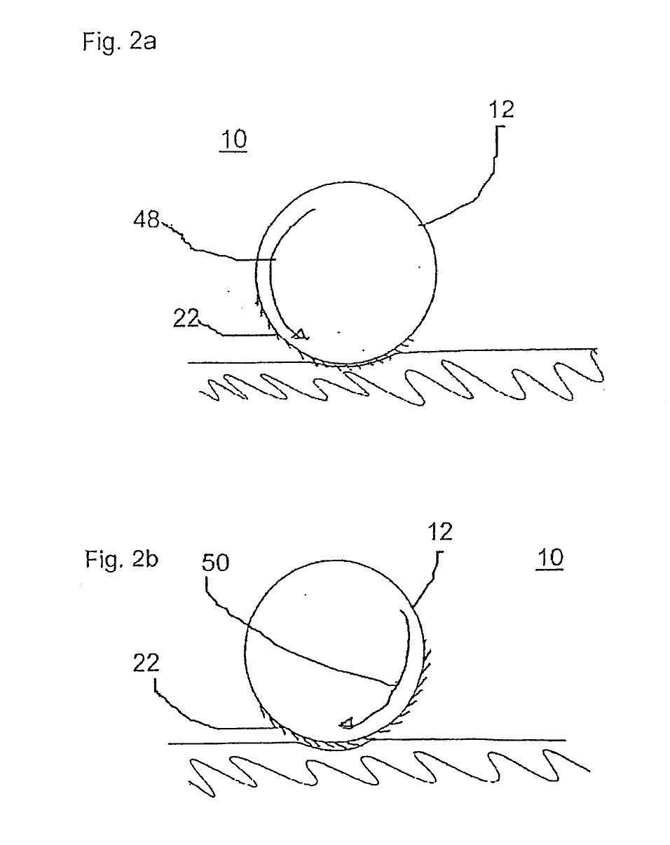

Reference is now made to FIGS. 2a and 2b. FIG. 2a is schematic cross-sectional view showing the penetration of microstructures 22 of microstructure roller system 10 of FIG. 1a when rolling element 12 is rolled in a first direction 48. FIG. 2b is schematic cross-sectional view showing the penetration of microstructures 22 of microstructure roller system 10 of FIG. 1a when rolling element 12 is rolled in a second direction 50. Microstructures 22 are configured by altering their shape and/or orientation on rolling element 12, such that when rolling element 12 is rolled in first direction 48, microstructures 22 penetrate through the surface of the skin to a first depth and when rolling element 12 is rolled in second direction 50, microstructures 22 penetrate through the surface of the skin to a second depth, the first depth being greater than the second depth. This effect is typically achieved by tilting microstructures 22 away from the radial direction of rolling element 12 until the above effect is achieved. Generally, microstructures 22 are oriented such that the second depth is zero. In other words, microstructures 22 do not penetrate, but simply massage, the skin when rolling element 12 is rolled in second direction 50. When rolling element 12 is rolled in first direction 48 penetration of microstructures 22 into the skin is generally enhanced. In accordance with a most preferred embodiment of the present invention, microneedles 22 have both a cutting edge and piercing tip oriented such that, the cutting edge and piercing tip enhance penetration into the skin.

Reference is now made to FIG. 3, which is a schematic view of integrally formed microstructure structures 18 of microstructure roller system 10 of FIG. 1a. Integrally formed microstructure structures 18 includes substrate 20 and microstructures 22. Substrate 20 has first major surface 24 and second major surface 26. Microstructures 22 project from second major surface 26. In accordance with a most preferred embodiment of the present invention each microstructure 22 has one or more hollows 52. However, it will be appreciated by those ordinarily skilled in the art that microstructures 22 can be formed without hollows 52, with or without through bores connecting first major surface 24 and second major surface 26. Hollows 52 are isolated from fluid connection with first major surface 24 of substrate 20. In other words hollow 52 is not a through bore connecting first major surface 24 and second major surface 26. Hollows 52 are configured such that, when microstructures 22 are inserted through the surface of the skin, the substance is transferred through the surface of the skin in hollows 52. Therefore, hollows 52 increase the substance transfer into the skin by increasing the surface area between the substance and the penetrated skin. Therefore, hollows 52 enhance penetration of active compounds into and through the skin, for example, but not limited to, for medical or cosmetic purposes. Hollows 52 are generally single-opening hollows. The term “single-opening hollow” is defined herein as, a hollow only having one opening on the surface of microstructure 22. It should be noted that hollows 52 can be implemented as “plural-opening” hollows, whereby each hollow has two or more openings on the surface of microstructure 22. It will be appreciated by those ordinarily skilled in the art that integrally formed microstructure structures 18 can be used with microstructure roller system 10 or with other microstructure systems such as injectors and applicators, for example, but not limited to whereby microstructure arrays are simply pushed or slid against the skin in various directions, for example, in a direction that enhances penetration of the microstructure tips and hollows. Additionally, it will be appreciated by those ordinarily skilled in the art that integrally formed microstructure structures 18 can be used for one time application or multiple uses. Also, it will be appreciated by those ordinarily skilled in the art that integrally formed microstructure structures 18 can be applied for a short period of time (equivalent to a bolus) or worn for many hours. The substance transferred by hollows 52 of microstructures 22 is typically a medical substance or a cosmetic substance, for example, but not limited to, drugs or cosmetic compounds for local or systemic distribution. It will be appreciated by those ordinarily skilled in the art that the substance can be for example, but not limited to a cream, liquid or gel. Additionally, the substance can be a dry substance such as a powder or dry film which is disposed in hollows 52. It is advantageous to formulate drugs into non-liquid forms (for example, but not limited to, dry films, powders, gels, cream and lyophilized drugs) for various reasons, for example, but not limited to, concentration, stability, biocompatibility and dose control. Such non-liquid formulations are difficult to deliver through the skin using conventional techniques. Syringes or other liquid relevant mechanisms do not provide a solution for non-liquid forms. These non-liquid substances can be used for example, but not limited to, multiple indications and molecular groups including the delivery of proteins and peptides (for example, in lyophilized form), vaccines, DNA vaccines, small molecules, large carbohydrate-based drugs and active cosmetic compounds. The term “medical” is defined herein as, a process having a primary purpose of improving or maintaining the state of health of a patient. The term “cosmetic” is defined herein as, a process that the sole or primary intended result is purely aesthetic. It will be appreciated by those ordinarily skilled in the art that the substance can be applied directly to the skin. In this scenario, the substance is disposed into hollows 52 as microstructures 22 are pushed into the skin.

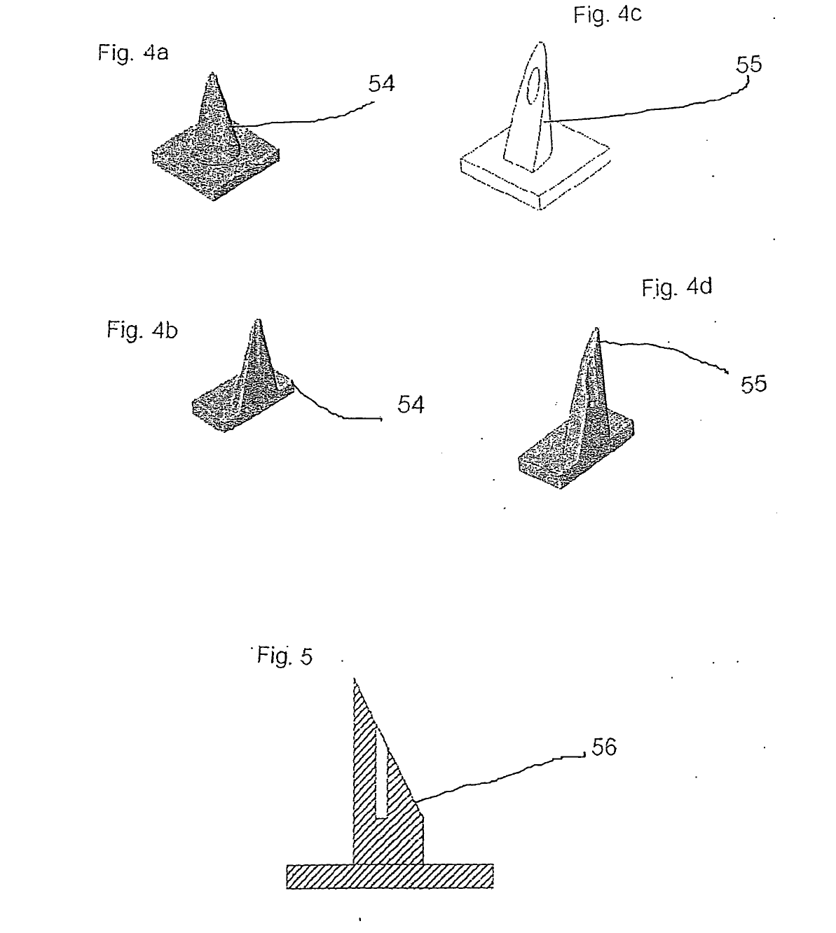

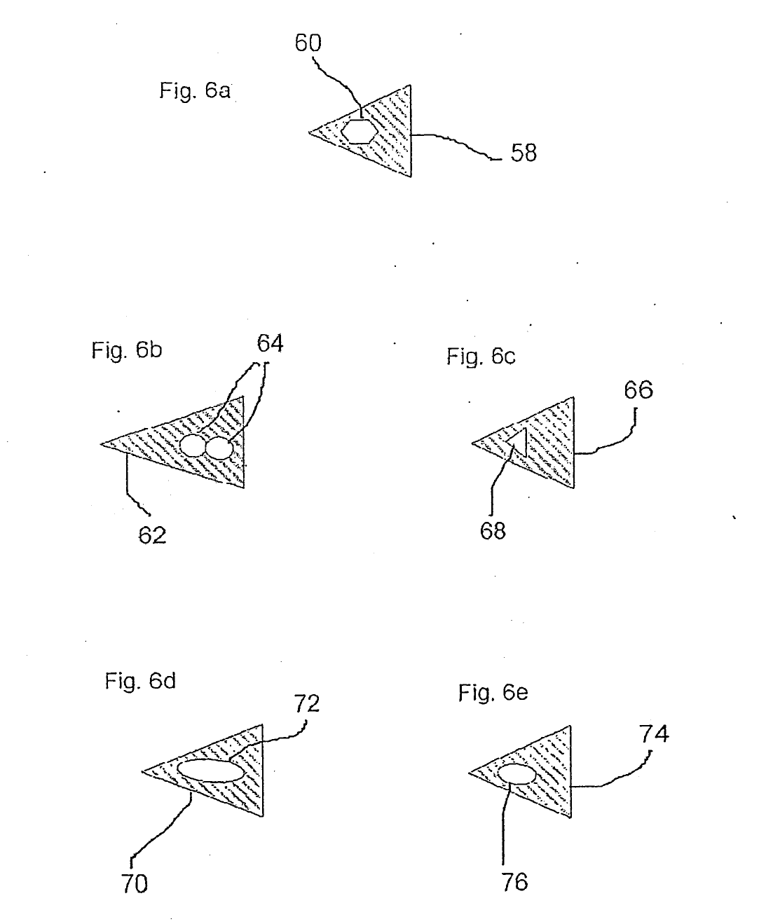

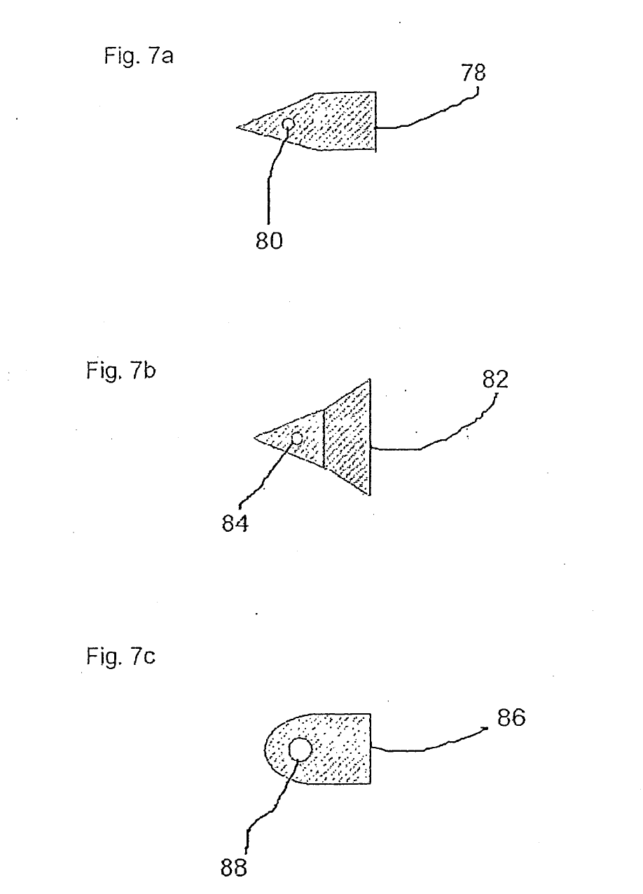

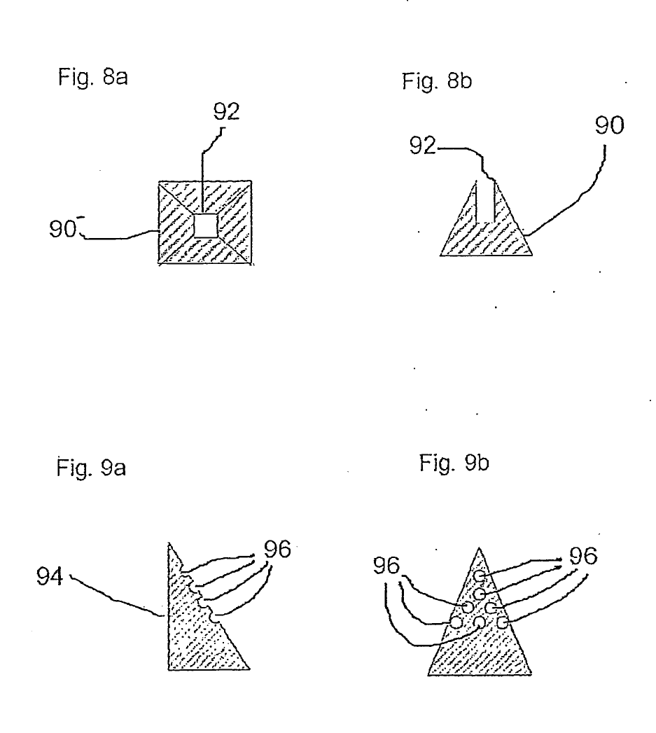

Reference is now made to FIGS. 4a to 9b, which are various views of “cupped” microstructures for use with microstructure roller system 10 of FIG. 1a. The term “cupped” microstructures is a term used to describe microstructures having hollows, because the hollows act like cups transferring a substance within the cups from outside of the skin to below the surface of the skin. It will be appreciated by those ordinarily skilled in the art that: (i) hollows can be formed having different cross-sections; (ii) hollows can be formed in different shaped microstructures; (iii) hollows can be formed at different locations within the microstructures; and (iv) hollows can be formed having different dimensions. It will be appreciated by those ordinarily skilled in the art that hollows can formed in microstructures using techniques known to those skilled in the art. For example, hollows are formed in silicon microstructures by selectively etching the silicon. For example, hollows are formed in polymer microstructures by selectively forming the polymer microstructures with one or more hollows using molding or lithographic techniques. Prior art techniques for forming microstructures are disclosed in pending patent applications, PCT Application No. IL03/00165 filed Mar. 4, 2003 and U.S. Application No. filed Mar. 27, 2003. and in U.S. Pat. Nos. 6,533,949 and 6,558,361 and a publication entitled “Process development for polymer needles by using SU-8 technology and silicon molding techniques” by Dominique Maria Altpeter of Mesa+ Institute, University of Twente, Enschede, Netherlands. It should be noted that consideration must be given for the overall strength of the microneedle versus the size of the hollow. If the hollow/microstructure ratio is too large then the structure may break on or during penetration. FIG. 4a is an isometric view of a cupped microstructure 54. FIG. 4b is a cut away view of microstructure 54 of FIG. 4a. FIG. 4c is an isometric view of an asymmetric cupped microstructure 55 for use with the microstructure roller system of FIG. 1a. FIG. 4d is a cut away view of asymmetric cupped microstructure 55 of FIG. 4c. FIG. 5 is a cross-sectional view of a cut cupped microstructure 56. FIG. 6a is a top view of a pyramidal microstructure 53 having a hollow 60 with a hexagonal cross-section. FIG. 6b is a top view of a pyramidal microstructure 62 having two hollows 64 of oval cross-section. FIG. 6c is a top view of a pyramidal microstructure 66 having a hollow 68 with a triangular cross-section. FIG. 6d is a top view of a pyramidal microstructure 70 having a wide hollow 72 with an oval cross-section. FIG. 6e is a top view of a pyramidal microstructure 74 having a narrow hollow 76 with an oval cross-section. FIG. 7a is a top view of a first microstructure 78 having a hollow 80 with a circular cross-section. FIG. 7b is a top view of a second microstructure 82 having a hollow 84 with a circular cross-section. FIG. 7c is a top view of a third microstructure 86 having a hollow 88 with a circular cross-section. FIG. 8a is a top view of a pyramidal microstructure 90 having a hollow 92 with a square cross-section. FIG. 8b is a cross-sectional view of pyramidal microstructure 90 of FIG. 8a. FIG. 9a is a cross-sectional view of a microstructure 94 having a plurality of hollows 96 thereon for use with the microstructure roller system of FIG. 1a. FIG. 9b is a side view of hollows 96 of FIG. 9a.

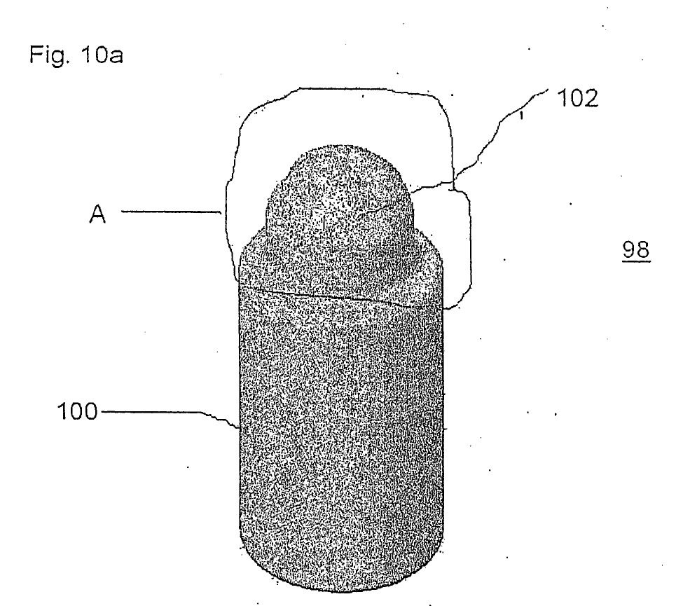

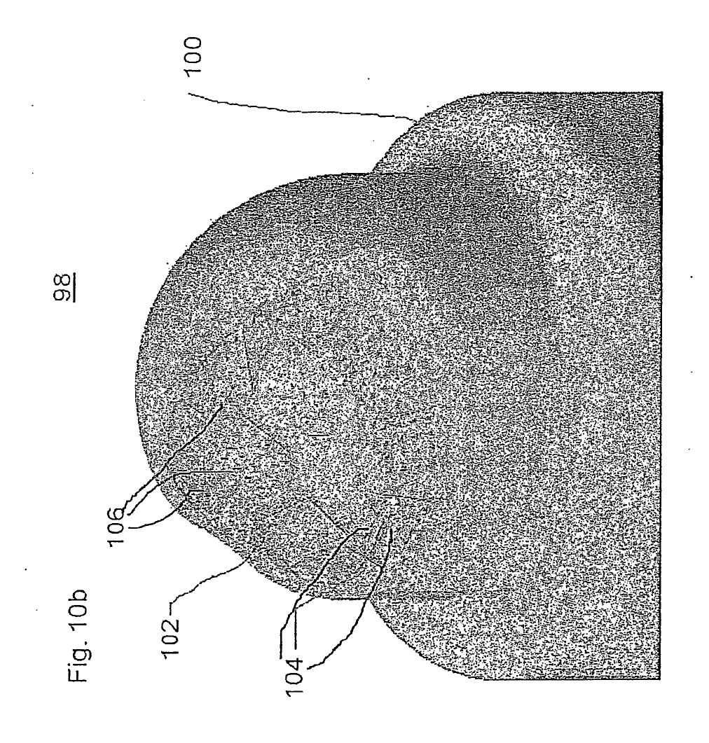

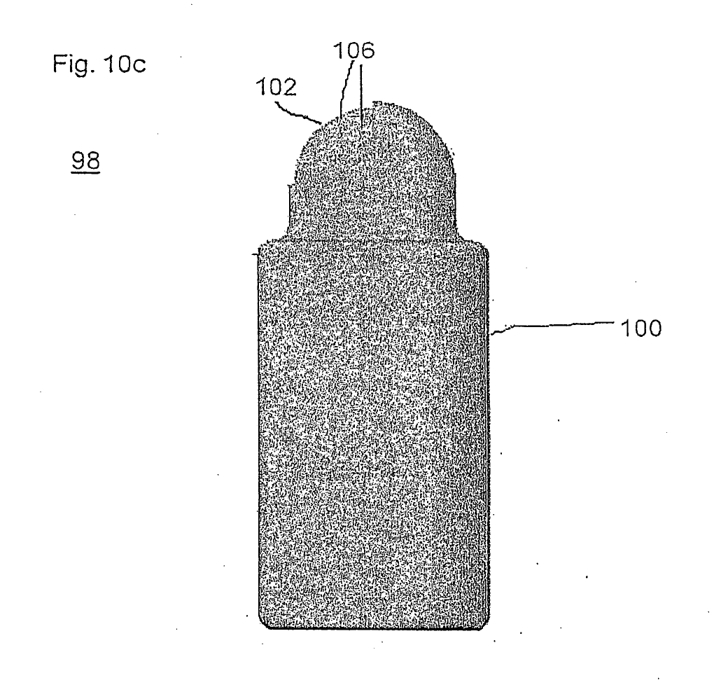

Reference is now made to FIGS. 10a to 10c which are various views of a microstructure ball-roller system 98 that is constructed and operable in accordance with a second alternate embodiment of the present invention. FIG. 10a is an isometric view of microstructure ball-roller system 98. FIG. 10b is an expanded view of the region marked by the letter A in FIG. 10a. FIG. 10c is a side view of microstructure ball-roller system 98. Microstructure ball-roller system 98 has a dispensing arrangement 100 and a rolling element 102. Rolling element 102 is substantially spherical. Microstructure ball-roller system 98 includes a plurality of substrates 106 having microstructures 104 thereon. Microstructures 104 are typically formed integrally with substrate 106. Substrates 106 are disposed on rolling element 102. It will be appreciated by those ordinarily skilled in the art that microstructures 104 can be formed integrally with rolling element 102. Rolling element 102 rotates within an end of dispensing arrangement 100. It will be appreciated by those ordinarily skilled in the art that the surfaces of dispensing arrangement 100 which come into contact with rolling element 102 and microneedles 104 need to be soft enough to ensure that microneedles 104 are not broken as rolling element 102 rotates. Nevertheless, these surfaces of dispensing arrangement 100 need to be robust enough to retain rolling element 102 in the end of dispensing arrangement 100. These surfaces are typically formed from a rubber material. Dispensing arrangement 100 is generally an elongated member with a circular cross-section. Dispensing arrangement 100 is hollow and is capable of storing a fluid substance (medical or cosmetic) which is dispensed onto microstructures 104. Microstructures 104 are preferably microstructures having hollows.

Microstructure roller system 10, microstructure roller system 34 and microstructure ball-roller system 98 are either used to prepare the skin prior to treatment with a cosmetic or medicine (pre-treatment), or to apply the cosmetic or medicine itself (treatment), or for use after the cosmetic or medicine has been applied (post treatment). This action improves the effective penetration of the treatment into the skin. For example, microstructure roller system 10 is used as a pre-treatment device which prepares the skin before applying a cream, like vitamin C containing cream, in order to increase the skin permeability and effectiveness of the cream in the skin. Applications, include, but are not limited to anti-aging, anti-wrinkle and anti-spotting treatment.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art which would occur to persons skilled in the art upon reading the foregoing description.

What is claimed is:

1. A microstructure structure for transferring a substance through the surface of the skin, comprising:
(a) a substrate having a first side and a second side; and
(b) a plurality of microstructures projecting from said second side of said substrate, each of said microstructure having at least one hollow, said hollows being isolated from fluid connection with said first side of said substrate, said hollows being configured such that, when said microstructures are inserted through the surface of the skin, at least part of the substance is transferred through the surface of the skin in said hollows.

2. The structure of claim 1, wherein said hollows is a single-opening hollows.

3. The structure of claim 1, wherein each of said microstructures has a plurality of hollows isolated from fluid connection with said first side of said substrate.

4. The structure of claim 1, further comprising a medical substance disposed in said hollows.

5. The structure of claim 1, further comprising a cosmetic substance disposed in said hollows.

6. The structure of claim 1, further comprising a rolling arrangement, wherein said rolling arrangement, said substrate and said microstructures together form a microstructure rolling system, said microstructure rolling system being configured such that, at least part of the substance is transferred through the surface of the skin as said microstructure rolling system is rolled over the surface of the skin.

7. The structure of claim 6, wherein said microstructure rolling system includes a dispensing arrangement configured to store the substance and to dispense the substance into said hollows of said microstructures.

8. A microstructure structure for transferring a substance through the surface of the skin, comprising:
(a) a substrate having a first side and a second side; and
(b) at least one microstructure projecting from said second side of said substrate, said at least one microstructure having at least one hollow, said at least one hollow being isolated from fluid connection with said first side of said substrate, said at least one hollow being configured such that, when said at least one microstructure is inserted through the surface of the skin, at least part of the substance is transferred through the surface of the skin in said at least one hollow.

9. The structure of claim 8, wherein said hollow is a single-opening hollow.

10. The structure of claim 8, wherein said at least one microstructure has a plurality of hollows, said hollows being isolated from fluid connection with said first side of said substrate.

11. A method for transferring a substance through the surface of the skin using a microstructure structure, the microstructure structure including a substrate having a first side and a second side, the microstructure structure including a plurality of microstructures, projecting from the second side of the substrate, the method including the steps of:
(a) applying the substance to the microstructure structure from said second side;
(b) disposing the microstructure structure onto the surface of the skin, such that the microstructures penetrate the surface of the skin thereby transferring at least part of the substance through the surface of the skin.

12. The method of claim 11, wherein:
(a) said step of applying is performed by disposing at least part of the substance into hollows of the microstructures, said hollows being isolated from fluid connection with the first side of the substrate; and
(b) said step of disposing the microstructure structure onto the surface of the skin is performed such that at least part of the substance is transferred through the surface of the skin in said hollows.

13. The method of claim 11, wherein the substance is a dry substance.

14. The method of claim 11, wherein the substance is a gel.

15. The method of claim 11, wherein the substance is a cream.

16. The method of claim 11, wherein the substance is a cosmetic substance.

17. The method of claim 11, wherein the substance is a medical substance.

18. A microstructure system for penetrating the surface of the skin, comprising:
(a) a rolling element;
(b) a plurality of microstructures interconnected with said rolling element, said microstructures having an inter-needle spacing of less than 1 mm; and
(c) a handle mechanically connected to said rolling element, said rolling element being configured to rotate with respect to said handle, wherein said rolling element, said microstructures and said handle are configured such that, when said handle is manipulated in order to roll said microstructures over the surface of the skin, the microstructures penetrate the surface of the skin.

19. A microstructure system for penetrating the surface of the skin, comprising:
(a) a rolling element;
(b) an integrally formed microstructure structure having a substrate and a plurality of microstructures projecting from said substrate, said microstructure structure being interconnected with said rolling element; and
(c) a handle mechanically connected to said rolling element, said rolling element being configured to rotate with respect to said handle, wherein said rolling element, said microstructure structure and said handle are configured such that, when said handle is manipulated in order to roll said microstructures over the surface of the skin, the microstructures penetrate the surface of the skin.

20. A microstructure system for penetrating the surface of the skin, comprising:
(a) a rolling element, said rolling element being substantially spherical;
(b) a plurality of microstructures interconnected with said rolling element; and
(c) a handle mechanically connected to said rolling element, said rolling element being configured to rotate with respect to said handle, wherein said rolling element, said microstructures and said handle are configured such that, when said handle is manipulated in order to roll said microstructures over the surface of the skin, the microstructures penetrate the surface of the skin.

21. A microstructure system for transferring a substance through the surface of the skin, comprising:
(a) a rolling element;
(b) a plurality of microstructures interconnected with said rolling element;
(c) a handle mechanically connected to said rolling element, said rolling element being configured to rotate with respect to said handle; and
(d) a dispensing arrangement mechanically connected to said rolling element, said dispensing arrangement being configured to store the substance and to dispenses the substance onto the microstructures, wherein said rolling element, said microstructures, said handle and said dispensing arrangement are configured such that, when said handle is manipulated in order to roll said microstructures over the surface of the skin, at least part of the substance is transferred through the surface of the skin.

22. A microstructure system for transferring a substance through the surface of the skin, comprising:
(a) a rolling element;
(b) a plurality of microstructures interconnected with said rolling element, each of said microstructures having a channel therethrough;
(c) a reservoir configured for storing the substance, said reservoir being interconnected with said channels of said microstructures such that, said reservoir is configured for dispensing the substance via said channels of said microstructures; and
(d) a handle mechanically connected to said rolling element, said rolling element being configured to rotate with respect to said handle, wherein said rolling element, said microstructures, said reservoir and said handle are configured such that, when said handle is manipulated in order to roll said microstructures over the surface of the skin thereby causing said microstructures to penetrate the surface of the skin, at least part of the substance is transferred through the surface of the skin via said channels of said microstructures.

23. A microstructure system for penetrating the surface of the skin, comprising:
(a) a roller;
(b) a handle mechanically connected to said roller, said roller being configured to rotate with respect to said handle; and
(c) a plurality of microstructures interconnected with said roller, said microstructures being configured such that:
(i) when said roller is rolled in a first direction said microstructures penetrate through the surface of the skin to a first depth; and
(ii) when said roller is rolled in a second direction said microstructures penetrate through the surface of the skin to a second depth, wherein said first depth is greater than said second depth.

24. The system of claim 23, wherein said second depth is zero.
